(19) United States
(12) Patent Application Publication
(10) Pub. No.: US 2025/0041005 A1
(43) Pub. Date: Feb. 6, 2025

(54) VISUALIZATION METHOD AND MEDICAL SYSTEM

(71) Applicant: Siemens Healthineers AG, Forchheim (DE)

(72) Inventors: Nils Pickert, Erlangen (DE); Reinier Kortekaas, Erlangen (DE)

(21) Appl. No.: 18/790,284

(22) Filed: Jul. 31, 2024

(30) Foreign Application Priority Data
Jul. 31, 2023 (DE) 10 2023 207 289.0

(51) Int. Cl. A61B 34/35; A61B 34/30; G16H 30/40; G16H 40/67

(52) U.S. Cl. CPC A61B 34/35; G16H 30/40; G16H 40/67; A61B 2034/302

(57) ABSTRACT

The invention relates to a method for visualizing an overshoot movement caused by a latency of a data transmission link with respect to a movement of a device component of a medical system, the movement being controlled by a person arranged remotely from the device component via the data transmission link, comprising the following steps: determination of the current latency of the data transmission link for controlling the movement, provision of a current speed of movement of the movement of the device component, determination of the length of the overshoot movement based on the current latency and the current speed of movement, and visualization of at least one expected position of the device component determined on the basis of the length of the overshoot movement using an image.

This application claims the benefit of German Patent Application No. DE 10 2023 207 289.0, filed on Jul. 31, 2023, which is hereby incorporated by reference in its entirety.

### BACKGROUND

The present embodiments relate to visualizing an overshoot movement caused by a latency of a data transmission link with respect to a movement of a device component of a medical system.

Typically, when performing an interventional medical procedure, a person treating or performing an operation is located with a patient on whom the interventional medical procedure is to be performed (e.g., at a patient table on which the object under investigation, such as the patient, is positioned).

In more recent applications, however, interventional medical procedures are also carried out in whole or in part by remote control or remotely. As a result, the person treating or performing an operation does not always have to be in situ with the object under investigation (e.g., the patient) in order to carry out such a procedure. Instead, the person may use a remote control unit to control a robotic system, for example, with which the interventional medical procedure is carried out on the object under investigation. The remote control unit may thus be spatially remote from the object under investigation. The remote control unit may therefore even be in another town, another country, or on another continent than the object under investigation. Between the remote control unit and the system to be controlled (e.g., the robotic system), there is a data transmission link for transmitting data (e.g., control commands and image data).

One problem with such long-range data transmission links is the so-called latency in the command chain. As a result, the actual reaction of the controlled system is delayed compared to the time of the control command. This results in the device component continuing to move for a while before the device component stops (e.g., an overshoot). For example, a movement of an object may be controlled via remote control, and the movement itself may be observed by a camera. If there is a delay of 5 ms in the data connection, the remotely located person sees the reaction of the object with a delay of 5 ms after the reaction has actually happened, and a command to stop the movement likewise reaches the system with a delay of 5 ms. In addition to the reaction time of the person, the object moves further than intended for twice 5 ms. This may be very critical (e.g., in medical applications, such as the movement of a catheter in the body of a patient).

Showing the latency of an online connection numerically on a display is known, for example, from the online gaming industry.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, a method that enables a person operating a medical system remotely from an object under investigation to reduce restrictions caused by a latency of the data transmission link is provided. As another example, a medical system suitable for carrying out the method is provided.

In one embodiment, a method for visualizing an overshoot movement to be expected due to a latency of a data transmission link to a movement of a device component of a medical system is provided. In another embodiment, a medical system is provided.

The method according to the present embodiments for visualizing an overshoot movement caused by a latency of a data transmission link with respect to a movement of a device component of a medical system includes determining the current latency of the data transmission link for controlling the movement. The movement is controlled by a person arranged remotely from the device component via the data transmission link. The method includes providing a current speed of movement of the movement of the device component, determining the length of the overshoot movement based on the current latency and the current speed of movement, and for the person arranged remotely, visualizing an expected position of the device component determined based on the length of the overshoot movement using an image.

This creates a visual aid for the operator, by which the operation of the medical system or the moving device component from a remote location (e.g., not only in another room, but even in another hospital, town, or even country) is significantly simplified. For example, a display is shown on a display unit located with the remote person. The person does not have to manually allow for the latency of a data transmission link themself, but is shown directly by an image, for example, where the device component is likely to move to even if the person gives an immediate stop command, or at least what effect the overshoot movement of the device component is likely to have. To do this, the medical system only needs the current latency and the current speed of movement of the device component, and from this, determines how far or to which position the device component will still move if the person stops the movement immediately. Taking into account the outward and return path (e.g., the image transmission and the transmission of the control command via the data transmission link), for example, the length Ü may be calculated as twice the latency L times the speed of movement v (e.g., Ü=2·L·v).

For example, a probability estimate may also be taken into account for the progress of the movement (e.g., length of the overshoot movement) or the expected position of the device component that is still to be expected due to the latency. The method according to the present embodiments may significantly reduce the risk of incorrect operation due to the person reacting too late, which leads to a minimization of risk for the patient. The visualization of the overshoot movement may be displayed, for example, as an overlay or superimposition or highlighting of the image. The expected positions of the device component may be displayed on the image faded in, faded over, or marked (e.g., as a rectangle, circle, or point). A warning may also be displayed (e.g., in color or shape) if an imminent danger is detected or is to be expected.

According to one embodiment, the image is a previously recorded image or a live image that was recorded by an imaging device associated with the medical system. The imaging device may be, for example, an X-ray device (e.g., a fluoroscopy device or an angiography device), a magnetic resonance device, or an ultrasound device. The imaging device may, for example, record images of the device component and/or the object under investigation. This is the case, for example, in the context of an instrument moved in the body of a patient for an interventional procedure or an operation. Alternatively, the device component itself may be part of the imaging device so that recorded images may indirectly display its movement. Instead of using a medical imaging device, images may also be recorded with one or more cameras, video cameras, or other optical sensors, for example.

According to a further embodiment, the device component is formed by an automatically or semi-automatically controlled instrument or a part thereof. The instrument may be arranged, for example, in or on an object under investigation. For example, the device component is formed by a catheter, a guide wire, or a guide wire tip that is arranged in a cavity organ (e.g., the object under investigation) of a patient. In many known interventional procedures, catheters or guide wires are moved forward semi-automatically by a person using a robotic system. The method helps to provide that this may also be carried out by a person arranged remotely (e.g., far away) without suffering any disadvantages due to a possible latency of the data transmission link. The progress of, for example, the guide wire or catheter or its tip through the cavity organ may be recorded by the imaging device (e.g., fluoroscopy device).

According to a further embodiment, the device component is part of the imaging device, and the visualization includes a display of the expected recording area based on the image. The device component may be, for example, a C-arm, an X-ray detector, or a collimator that moves or is adjusted. If, for example, the X-ray detector moves closer to the object under investigation, the recording area changes; in this case, the expected recording area may be shown in the image using the current or a previously recorded image (e.g., as a faded in or marked square). The visualization of the positions is therefore not carried out directly, but indirectly by displaying the expected recording area.

According to one embodiment, the visualization includes one or more expected positions of the device component on the image, which is displayed on a display unit for the person arranged remotely. For example, the expected positions of the device component are shown as a rectangle, a circle, or a point on the image. For example, if the movement of a guide wire/a guide wire tip or a catheter in a cavity organ is considered, a circle may be faded in, mapped, or inserted as a marker on the previous or current image, which indicates the position in the cavity organ to which the guide wire/the guide wire tip or the catheter will continue to move when stopped immediately.

According to a further embodiment, the expected length of the overshoot movement is calculated or estimated with regard to a predetermined probability. For example, additional factors may be taken into account (e.g., fluctuations in the latency or the speed of movement or changes in direction or other interference). For the predetermined probability, for example, 80%, 90%, or 95% may be used.

According to a further embodiment, the length of the overshoot movement and/or the expected position of the device component is determined by including the planning path of the device component and/or image information from previously recorded images and/or information of the object under investigation. If, for example, an intervention instrument is located in a cavity organ, the planned path may be included, for example, to the effect that it is assumed that the instrument remains on the planned path during the overshoot movement. Alternatively, it may also be assumed, for example, that the instrument is inevitably located inside the cavity organ. Possible positions may then be determined, for example, by a segmented image (e.g., pre-op, previously recorded image). It is also possible, for example, to indicate in color whether the device component is still on the planning path (e.g., green) or is already moving beyond the planning path (e.g., red). Probabilities may also be used to determine the expected positions (e.g., 80%, 90%, or 95%).

According to a further embodiment, the method is repeated a number of times or continuously (e.g., until an abort criterion is reached). In this way, the remotely controlling person may always see the currently expected overshoot distance or position of the device component and receives continuous updates. The abort criterion may be, for example, a stopping of the movement of the device component.

According to a further embodiment, the current latency itself is additionally displayed graphically or numerically (e.g., on a display for the remotely located person). In this way, the controlling person may obtain further information about the latency and use the further information for control accordingly.

According to a further embodiment, at least one trigger time is calculated and displayed, taking into account the latency, in the event of a triggering action to be triggered in a synchronized manner by the person located remotely. In this case, it is not the trigger time for an on-site triggering action that is displayed, but a latency-delayed trigger time that the remotely located person may use for control in order to start the triggering at the right time. This may be used, for example, in the case of a triggering action that is coordinated with a cyclical movement such as the heartbeat (e.g., recording of X-ray images). In this way, it is also possible for a remotely controlling person to easily find the optimum trigger time.

The present embodiments also include a medical system for carrying out a method described above, including in situ or at the object under investigation: a movable device component; an imaging device for recording images; a system controller for controlling the medical system and the device component and for providing the speed of movement of the device component; a calculation unit for determining the length of the overshoot movement based on the current latency and the current speed of movement; and a unit for providing the respective current latency. The calculation unit and the unit for providing the respective current latency may be formed by one or more processors. The medical system also includes, arranged remotely from the device component: a remote control unit for operating at least the movable device component by a person; a display unit for visualizing the expected position of the device component using an image; and a data transmission link between the remote control unit and the medical system.

According to one embodiment, the medical system includes a robotic system for moving an instrument through a cavity organ of a patient. The device component is formed by the instrument. The instrument may be, for example, a guide wire, a catheter, a stent, an intervention tool, or something similar that is moved by the robotic system (e.g., via a drive) semi-automatically controlled via the remote control unit.

### BRIEF DESCRIPTION OF THE DRAWINGS

FIG. 1 shows a sequence of acts of a method for visualizing an overshoot movement;

FIG. 2 shows a sequence of acts of a further method for visualizing an overshoot movement;

FIG. 3 is a view of an image with a position of a guide wire tip to be expected due to an overshoot movement generated by a latency;

FIG. 4 shows three views of images with expected recording areas for three different latencies when moving a C-arm;

FIG. 5 is a view of a movement of the C-arm;

FIG. 6 is a representation of an image of an ECG of a cyclical cardiac movement with a marked trigger period; and

FIG. 7 is a view of a medical system and a person operating remotely via a data transmission link.

### DETAILED DESCRIPTION OF THE DRAWINGS

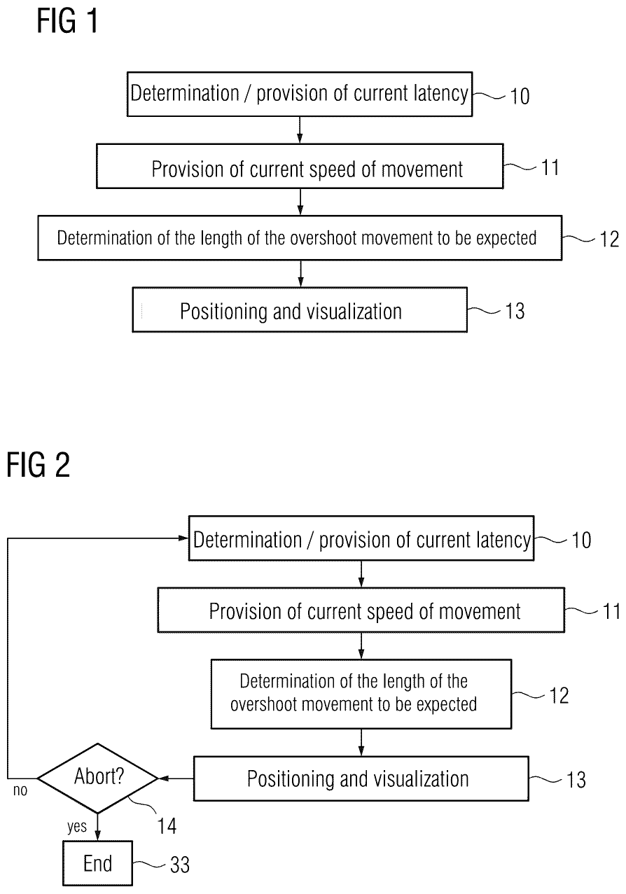

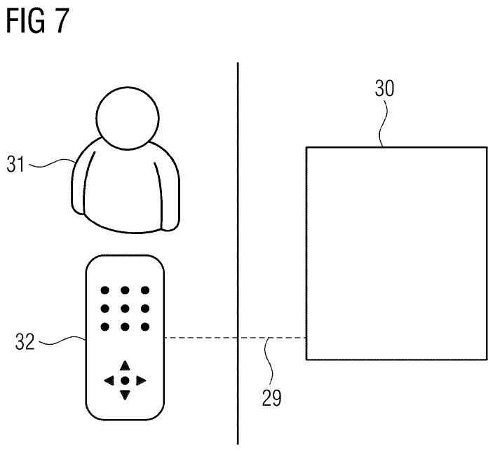

FIGS. 1 and 2 each show sequences of acts of a method for visualizing an overshoot movement caused by a latency of a data transmission link with respect to a movement of a device component of a medical system. The method relates to medical systems 30 in which at least one device component is controlled by a person 31 arranged remotely from the device component via a remote control unit 32 via the data transmission link 29 (see FIG. 7). Remote may be that the person is not in sight, but at a different location to the medical system and the object under investigation (e.g., in another hospital, in another town, another country, or even on another continent). The person arranged remotely also has a display unit on which, for example, images or other information are displayed. This may be a monitor or a touchpad, for example.

The medical system 30 may include, for example, an imaging device such as, for example an X-ray device, and/or a robotic system for navigating an instrument in the body of a patient to perform an interventional procedure. The two main examples of an application of the method described here are a remotely controlled positioning of a C-arm of an X-ray device and a remotely controlled movement of a catheter or guide wire in a cavity organ of the patient. The moved device component is the C-arm in the first case, and the moved component is the catheter or guide wire in the second case. Robotic systems, by which the semi-automatic advance of a catheter or guide wire, for example, may be effected in a cavity organ of a patient with robotic support are known, for example, from EP 3406291 B1.

However, the method may also be used for a number of other applications and device components of a medical system.

In a first act 10, a current latency of the data transmission link is provided (e.g., by determining or measuring the current latency). This may be carried out using known methods. For example, control units may be continuously sent back and forth between the remote control unit and the medical system in order to always have a current value for the latency L. In a second act 11, the current speed of movement v of the device component (e.g., the instrument or catheter or the C-arm) is provided. The information may be provided or also measured by the medical system, for example. For example, in the case of a guide wire or catheter moved by a robotic system, the feed rate caused by the drive, for example, may be provided and used, or a speed of movement determined from live imaging using image recognition may also be used. Values provided by the drives or otherwise measured (e.g., by a positioning system or other sensors) may also be used for the movement of the C-arm. It may also be provided that individual direction components of the speed are determined/measured or made available in principle.

In a third act 12, the length of the overshoot movement or how far or to which position the device component will continue to move if the person stops the movement immediately is determined or estimated based on the current latency and the current speed of movement.

In the simplest case, for example, the length Ü may be calculated as twice the latency L times the speed of movement v (e.g., Ü=2·L·v) taking into account the outward and return path via the data transmission link (e.g., the image transmission from the image recording system to the remotely arranged display unit, and the transmission of the control command from the remotely arranged remote control unit to the medical system).

In some cases, only one directional component of the speed may be used (e.g., if a risk or relevance exists only for a certain direction). Accordingly, different directional components may also be determined and used individually. Probabilities or estimates may also be included (e.g., whether and to what extent the speed of movement or the latency is subject to fluctuations). Previously recorded values, for example, may be used for this purpose. For example, it may be calculated that statistically a short-term fluctuation in latency of, for example, 20%, 10%, or 5% may occur.

If there are no exact values for latency and speed of movement, estimates may also be used. In addition, the respective braking distance of the device component may also be taken into account (e.g., when moving the C-arm). This may be added to the result, for example, to determine the length. In addition, other factors may also be taken into account (e.g., interference or other detected irregularities). For example, when moving an instrument through a vascular system, bifurcations or characteristics of the vascular systems may be taken into account, or mechanical characteristics of the device may be taken into account for a moving C-arm.

In a fourth act 13, at least one position of the device component that is determined based on the length of the overshoot movement and is to be expected in the event of an immediate triggering of a control signal via the data transmission link is then visualized using an image. This may be a direct or indirect representation of the expected position or positions.

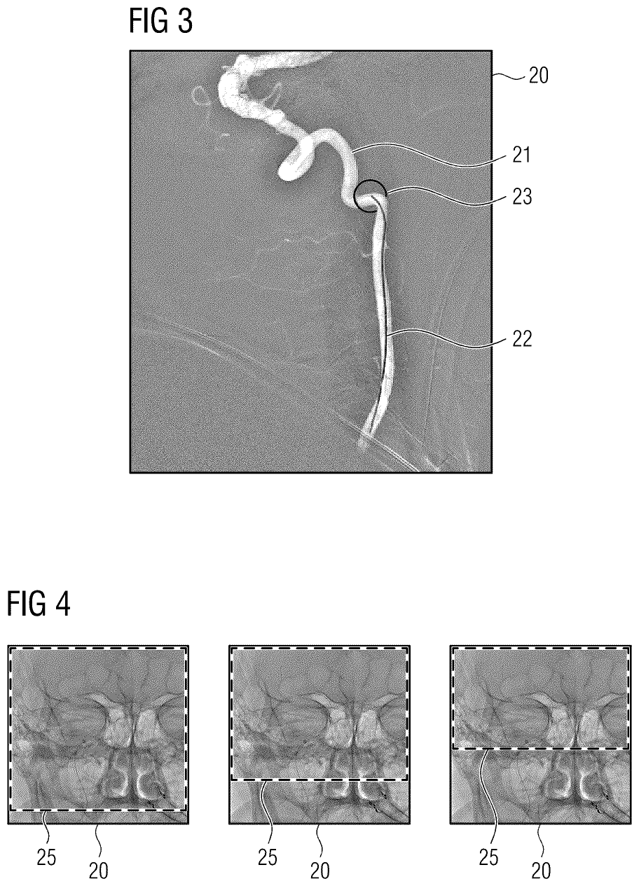

A direct representation may be, for example, the expected position or a number of positions based on a previously recorded image of the device component, such as, for example, in the case of a catheter moved by a robotic system. In one embodiment, a previous or current live image may be used, processed by image recognition or segmentation, and the expected position may be inserted into the previous or current live image via fading in/fading over, highlighting, or a virtual image. A number of positions may also be shown by a marked area or a drawn circle. For example, FIG. 3 shows an image 20 (e.g., X-ray image) of a guide wire 22 that is being moved in a vessel 21 of a patient. A circle 23 that is faded into the image 20 indicates how far the tip of the guide wire 22 is likely to move due to the latency of the data transmission link.

The expected positions of the device component may also be determined, for example, by including the planning path of the guide wire or catheter or by using image information from one or more previously recorded images and/or with the aid of information about the object under investigation. If, for example, an instrument is located in a cavity organ, it may also be assumed, for example, that the instrument remains on the planned path during the overshoot movement. Alternatively, it may also be assumed, for example, that the instrument is inevitably located at least within the cavity organ. Possible positions may then be determined, for example, by a segmented image (e.g., pre-op, previously recorded image). It is also possible, for example, to use a correspondingly adjusted color to indicate whether the device component is still within the planning path (e.g., green) or is already moving beyond the planned path (e.g., red) due to the overshoot movement. When determining the positions to be expected, estimates of probabilities may also be used so that, for example, the area in which the device component is located is marked with a probability of 80%, 90%, or 95% due to the overshoot movement.

An indirect representation may be used, for example, when adjusting a recording system and, for example, a C-arm 24 relative to the object under investigation 26 (for an example of a movement, see FIG. 5 in the direction of the arrow). The person located remotely is shown an image 20 of a current recording area (e.g., fluoroscopy) recorded by the X-ray detector, for example, on their display unit, which is also arranged remotely. In addition to determining the length of the overshoot movement, the estimated position of the expected recording area 25 is also determined accordingly and displayed relative to the current recording area on the current image 20 (see FIG. 4; for three different latencies, a low latency on the image 20 on the left, a medium latency on the image in the center, and a high latency on the image on the right).

For the determination of the expected recording areas (e.g., also relative to the current recording area on the image), for example, the recording geometry of the C-arm, further information, and measurements (e.g., position determination, etc.) may be used. The expected recording area 25 is indicated on the image 20 (e.g., current or most recent image with or without X-rays), for example, by a frame. The expected recording area 25 may be displayed incompletely, for example, if the movement leads out of the current recording area. A virtual image may also be used in addition to the recorded image (e.g., as a combination) or alternatively to display the expected recording area. Depending on the level of latency, certain colors may also be used; for example, the frame may be colored (e.g., red for high latency, yellow for medium latency, and green for low latency).

The latency itself may also be displayed as a numerical value or graphical element.

The method may, for example, be repeated continuously during remote control by the person located remotely, and visualization may be renewed accordingly. This is shown in FIG. 2, the method being repeated until an abort criterion 14. An abort criterion may represent, for example, a termination of operation or an abort command by the person.

The method creates a visual aid for the person arranged remotely, which significantly simplifies operation. The person is shown directly by an image where the device component is likely to move to even if the person gives an immediate stop command, or at least what effect is to be expected as a result of the overshoot movement of the device component. The visual representation may include a predicted range within which the device component will stop with a predetermined probability, taking into account possible fluctuations in the latency of the data transmission link.

In concrete terms, the method may also be as follows. For example, a catheter is controlled remotely and semi-automatically by a person with the aid of a robot. The catheter tip is detected on the current X-ray image, for example, by image recognition. Based on the speed of movement of the catheter, which is reported by the robot, and the measured latency of the data transmission link, the expected stopping point of the catheter is determined and displayed as a semicircle in front of the catheter tip. The expected path of the catheter through the cavity organ can be roughly estimated and displayed, for example, based on the direction of movement. It is also possible to take the course of the vessel into account when determining the stopping point. This may be available, for example, based on previous segmentation or image analysis from images with contrast agent and may be used to limit the visualization to the stopping point within the actual vessel and possibly even include possible foreshortening of the vessel. In addition, when determining expected positions of the catheter, it may also be taken into account that a sudden forward movement of the catheter may occur, for example, due to torsion or bending of the catheter within the vessel.

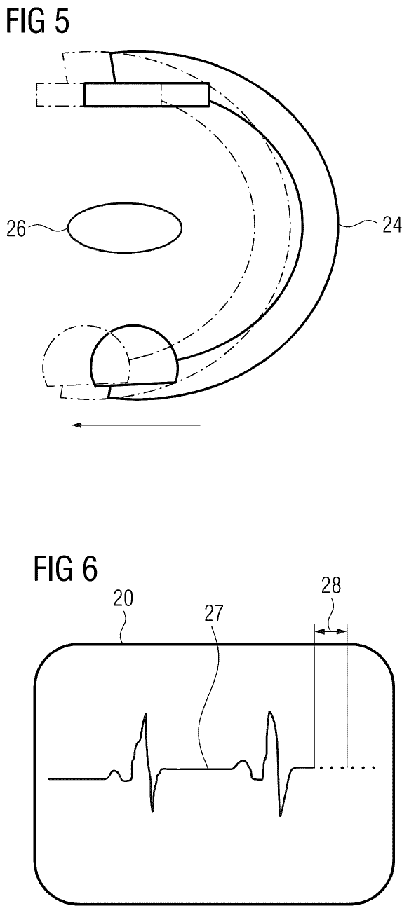

The method may be extended. For example, a person arranged remotely wants to trigger a device function (e.g. triggering X-rays) with the remote control unit, which is to be triggered synchronized with a certain cyclical movement (e.g., a cardiac movement or a breathing movement). For this purpose, the time intervals at which the triggering is to be initiated may be visualized on the display unit, taking into account the latency. This may be done, for example, by displaying a timeline with correspondingly marked time intervals. The measured ECG signals 27 (e.g., in the case of cardiac movement) may also be shown with the time intervals 28 (see FIG. 6). In this way, the person receives a trigger for initiating the function, which increases the probability that the synchronization may be triggered according to the cardiac phase. A similar application may also be provided for the use of instructions to the patient (e.g., breathing instructions).

Independent of the grammatical term usage, individuals with male, female, or other gender identities are included within the term.

The present embodiments may be briefly summarized as follows. For particularly intuitive remote control, a method for visualizing an overshoot movement caused by a latency of a data transmission link with respect to a movement of a device component of a medical system is provided. The movement is controlled by a person arranged remotely from the device component via the data transmission link. The method includes determining a current latency of the data transmission link for controlling the movement, providing a current speed of movement of the movement of the device component, determining a length of the overshoot movement based on the current latency and the current speed of movement, and visualizing an expected position of the device component determined based on the length of the overshoot movement using an image.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for visualizing an overshoot movement caused by a latency of a data transmission link with respect to a movement of a device component of a medical system, wherein the movement is controlled by a person arranged remotely from the device component via the data transmission link, the method comprising:
determining a current latency of the data transmission link for controlling the movement;
providing a current speed of movement of the movement of the device component;
determining a length of the overshoot movement based on the current latency and the current speed of movement; and
visualizing at least one expected position of the device component determined based on the length of the overshoot movement using an image.

2. The method of claim 1, wherein the image is a previously recorded image or a live image that was recorded by an imaging device belonging to the medical system.

3. The method of claim 1, wherein at least part of the device component, the object under investigation, or the device component and the object under investigation is shown on the image.

4. The method of claim 1, wherein the visualizing comprises displaying one or more expected positions of the device component on the image.

5. The method of claim 1, further comprising calculating an expected length of the overshoot movement with regard to a predetermined probability.

6. The method of claim 1, wherein the device component is formed by an automatically or semi-automatically controlled instrument.

7. The method of claim 6, wherein the device component is formed by a catheter or a guide wire.

8. The method of claim 2, wherein the device component is part of the imaging device, and the visualizing comprises displaying an expected recording area based on the image.

9. The method of claim 4, wherein the expected positions of the device component on the image are displayed faded in, faded over, or marked.

10. The method of claim 9, wherein the expected positions of the device component on the image are displayed marked as a rectangle, a circle, or a point.

11. The method of claim 1, wherein the length of the overshoot movement, the expected position of the device component, or the length of the overshoot movement and the expected position of the device component are determined taking into account a planning path of the device component, image information from previously recorded images, information of the object under investigation, or any combination thereof.

12. The method of claim 1, wherein the method is repeated a number of times or continuously, in particular until an abort criterion is reached.

13. The method of claim 12, wherein the method is repeated until an abort criterion is reached.

14. The method of claim 1, wherein the latency is also displayed graphically or numerically.

15. The method of claim 1, further comprising, in case of a triggering action to be triggered in a synchronized manner by the person arranged remotely, calculating at least one trigger time taking into account the latency, and displaying the at least one trigger time.

16. A medical system operable to visualize an overshoot movement caused by a latency of a data transmission link with respect to a movement of a device component of the medical system, wherein the movement is controlled by a person arranged remotely from the device component via the data transmission link, the medical system comprising:
a movable device component;
an imaging device operable to record images;
a system controller configured to:
control the medical system and the movable device component; and
provide a speed of movement of the movable device component;
a remote control unit arranged remotely from the movable device component for operating at least the movable device component by a person;
the data transmission link between the remote control unit and the medical system;
a calculation unit configured to determine a length of the overshoot movement based on a current latency and a current speed of movement;
a unit configured to provide the respective current latency; and
a remotely arranged display unit operable to visualize an expected position of the movable device component using an image.

17. The medical system of claim 16, further comprising a robotic system operable to move an instrument through a cavity organ of a patient,
wherein the movable device component is formed by the instrument.

18. The medical system of claim 16, wherein the imaging device operable to record live images.
